(19) United States
(12) Patent Application Publication
(10) Pub. No.: US 2026/0199362 A1
(43) Pub. Date: Jul. 16, 2026

(54) VITAMIN B2 FOR USE IN IMPROVING GUT HEALTH

(71) Applicant: DSM IP Assets B.V., Maastricht (NL)

(72) Inventors: Thanh-Van PHAM, Reinach (CH); Wilbert SYBESMA, Kaiseraugst (CH); Robert STEINERT, Kaiseraugst (CH); Ateequr REHMAN, Kaiseraugst (CH)

(21) Appl. No.: 18/872,488

(22) PCT Filed: Jun. 8, 2023
(86) PCT No.: PCT/EP2023/065386

§ 371 Date: Dec. 6, 2024

(30) Foreign Application Priority Data
Jun. 10, 2022 (EP) 22178541.3

(51) Int. Cl. A61K 31/525; A61K 35/747; A61P 1/00

(52) U.S. Cl. CPC A61K 31/525; A61K 35/747; A61P 1/00

(57) ABSTRACT

The present invention relates to vitamin B2 (riboflavin) for use in improving gut health in animals and humans. In particular, it was found that vitamin B2, upon delivery to the large intestine, increases the abundance of the beneficial bacteria Lactobacillus rhamnosus in the intestinal tract.

### FIELD OF THE INVENTION

The present invention relates to vitamin B2 (riboflavin) for use in improving gut health in animals and humans. It was found that vitamin B2, upon delivery to the large intestine, increases the abundance of the beneficial bacteria Lactobacillus rhamnosus in the intestinal tract.

### BACKGROUND OF THE INVENTION

Increasing evidence indicates that imbalances in the human gut microbiota (also referred to as “dysbiosis”) may be associated with Western diseases, including obesity and type 2 diabetes, as well as cardiovascular, autoimmune, and intestinal inflammatory disease. Thus, targeted modulation of the human gut microbiome intended to restore imbalances represents a potential therapeutic and preventive strategy and has attracted the attention of academics as well as those engaged in various industries. Public awareness and acceptance of substances that modulate the human gut microbiome continue to grow.

There is a consensus that certain live microorganisms, so called probiotics, have beneficial effects on human health. Lactobacillus rhamnosus GG (L. rhamnosus GG) was the first strain of the genus Lactobacillus to be patented in 1989, thanks to its ability to survive and to proliferate at gastric acid pH and in medium containing bile, and to adhere to enterocytes. Furthermore, L. rhamnosus GG is able to produce a biofilm that can mechanically protect the mucosa and different soluble factors beneficial to the gut by enhancing intestinal crypt survival, diminishing apoptosis of the intestinal epithelium, and preserving cytoskeletal integrity. L. rhamnosus GG inhibits some pathogens such as Salmonella species due to its lectin-like protein 1 and 2. Further, L. rhamnosus GG is able to promote type 1 immune-responsiveness by reducing the expression of several activation and inflammation markers on monocytes and by increasing the production of interleukin-10, interleukin-12, and tumor necrosis factor-α in macrophages. A large quantity of research data on L. rhamnosus GG proved its beneficial role in human health (Capurso, J Clin Gastroenterol. 2019 March; 53 Suppl 1: S1-S41). Lactobacillus rhamnosus was recently renamed Lacticaseibacillus rhamnosus.

It was demonstrated that vitamins may modulate the human gut microbiome. WO2020/043797 discloses that vitamins can be useful to increase the growth of certain beneficial bacteria in the intestine. However, the human gut is home to hundreds of different microbes, and it would be desirable to be able to boost specific beneficial bacteria. In particular, it would be desirable to increase the abundance of Lactobacillus rhamnosus in the intestine to enhance wellness, improve digestive health, and support the immune system.

### SUMMARY OF THE INVENTION

The present invention relates to the following items:
1) Vitamin B2 for use in increasing the abundance of Lactobacillus (Lacticaseibacillus) rhamnosus in the intestine of an animal, preferably a human, wherein said use comprises delivering the vitamin B2 to the large intestine.
2) Vitamin B2 for the use according to item 1, wherein the vitamin B2 is delivered to the large intestine by a delayed-release formulation.
3) Vitamin B2 for the use according to item 1 or 2, wherein said use comprises administering to the animal a formulation comprising a vitamin B2 dose of up to 200 mg/day, preferably 5-100 mg/day, more preferably 10-50 mg/day.
4) Vitamin B2 for the use according to any one of items 1-3, wherein Lactobacillus (Lacticaseibacillus) rhamnosus is co-administered with vitamin B2.
5) Vitamin B2 for the use according to any one of items 1-4, wherein the animal, including a human, is experiencing a condition selected from the group consisting of: gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, and cancer.
6) Vitamin B2 for the use according to any one of items 1-5, wherein the Lactobacillus (Lacticaseibacillus) rhamnosus is a Lactobacillus (Lacticaseibacillus) rhamnosus GG, preferably Lactobacillus rhamnosus DSM 32550.
7) An oral formulation comprising vitamin B2 and Lactobacillus (Lacticaseibacillus) rhamnosus.
8) The oral formulation according to item 7 which is a delayed-release formulation
9) A method of increasing the abundance of Lactobacillus (Lacticaseibacillus) rhamnosus in the intestine of an animal, preferably a human, comprising administering to the animal a formulation comprising an effective dose of vitamin B2.
10) The method according to item 9, wherein the animal is a human and the vitamin B2 is delivered to the large intestine.
11) The method according to item 9 or 10, wherein the vitamin B2 is delivered by a delayed-release formulation.
12) The method according to any one of claims 9-11, wherein Lactobacillus (Lacticaseibacillus) rhamnosus is co-administered with vitamin B2.
13) The method according to any one of items 9-12, which is a method of treating, preventing, or lessening the symptoms of gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, and/or cancer in an animal, including a human, in need thereof.
14) The method according to any one of items 9-13, wherein the Lactobacillus (Lacticaseibacillus) rhamnosus is a Lactobacillus (Lacticaseibacillus) rhamnosus GG, preferably Lactobacillus rhamnosus DSM 32550.

### BRIEF DESCRIPTION OF THE DRAWINGS

FIG. 1 shows the Lactobacillus rhamnosus GG levels (putative phage tail protein gene copies/mL) (±stdev) following 48 h of colonic incubation, averaged over six different human donors. Average results are shown for Lactobacillus rhamnosus GG alone or when co-supplemented with vitamin B2. Statistically significant differences between the test conditions and the control are indicated with ‘*’ (p<0.05).

### DETAILED DESCRIPTION OF THE INVENTION

Lactobacillus (Lacticaseibacillus) rhamnosus is a bacterial strain known for its beneficial effects on human health, and gut health in particular. The present inventors have found that vitamin B2 can boost the growth of Lactobacillus rhamnosus, leading to an increase of Lactobacillus rhamnosus levels in the gut.

Hence, in a first aspect, the present invention relates to vitamin B2 for use in improving gut health in an animal. Said improvement comprises or consists of increasing the abundance of Lactobacillus (Lacticaseibacillus) rhamnosus in the intestine of said animal. Specifically, the vitamin B2 is for use in increasing the abundance of Lactobacillus rhamnosus in the large intestine (colon) of an animal, and preferably said use comprises delivering the vitamin B2 to the large intestine. Preferably, the animal is a human.

Vitamin B2 (also known as riboflavin) is one of the water-soluble B vitamins which is an essential component of two major coenzymes, flavin mononucleotide (FMN; also known as riboflavin-5′-phosphate) and flavin adenine dinucleotide (FAD). These coenzymes play major roles in energy production; cellular function, growth, and development; and metabolism of fats, drugs, and steroids. Riboflavin can be purchased from DSM GmbH. Alternative suppliers are TER Chemicals Distribution Group, BIOCHEM Bernburg GmbH, DVA International GmbH, Falken Trade GmbH, and Neupert Ingredients GmbH.

The most common Lactobacillus (Lacticaseibacillus) rhamnosus strain is Lactobacillus rhamnosus GG. It can be purchased, for example, from Chr. Hansen, Denmark, as LGG®. Lactobacillus (Lacticaseibacillus) rhamnosus DSM 32550 (Biocare Copenhagen, Denmark) has a genomic sequence which is 99.99% identical to the genomic sequence of LGGR. It can thus be considered that L. rhamnosus DSM 32550 is identical or equivalent to LGGR for practical purposes. Therefore, L. rhamnosus DSM 32550 will herein be referred to as a “Lactobacillus rhamnosus GG”.

Alternative Lactobacillus rhamnosus strains are, inter alia, Lactobacillus rhamnosus HN001 (Howaru; Danisco/DuPont), Lactobacillus rhamnosus GR-1® (Chr. Hansen, Denmark), and Lactobacillus rhamnosus Rosell-11 (Lallemand, Canada).

Lactobacillus (Lacticaseibacillus) rhamnosus DSM 32550 (Biocare Copenhagen) is a preferred strain according to the present invention. It has been deposited at Leibniz Institute DSMZ-German Collection of Microorganisms and Cell Cultures, Inhoffenstr. 7B, D-38124 Braunschweig, Germany, according to the Budapest Treaty on 6. July 2017. The accession number given by the International Depository Authority is DSM 32550.

To achieve an increase of the abundance of Lactobacillus rhamnosus in the large intestine, the vitamin B2 is preferably directly delivered to the large intestine. That is, the vitamin is delivered/administered in a manner such that the vitamin is not absorbed in the stomach and/or small intestine; rather the vitamin is delivered/administered to the distal intestinal tract, preferably the large intestine (colon). This is preferably done by delivering/administering the vitamin B2 in a delayed-release formulation. Oral administration is preferred.

Preferably, the vitamin B2 dose administered to the animal is up to 200 mg/day, preferably 5-100 mg/day, more preferably 10-50 mg/day. In one embodiment, vitamin B2 is administered in an amount such that its local concentration in the colon is at least 0.001 g/L, preferably at least 0.01 g/L more preferably at 0.02 g/L. Preferred local concentrations in the colon range from about 0.001 g/L to about 0.5 g/L, or from about 0.005 g/L to about 0.2 g/L, preferably about 0.01 to about 0.02 g/L.

The probiotic strain Lactobacillus rhamnosus may be co-administered with the vitamin B2. Hence, the invention also relates to vitamin B2 for uses in increasing the abundance of Lactobacillus rhamnosus in the intestine of an animal, preferably a human, wherein said uses comprise delivering both the vitamin B2 and Lactobacillus rhamnosus to the large intestine. This can be done, for example, if Lactobacillus rhamnosus is not yet colonizing the intestine, or is present only in low numbers. Lactobacillus rhamnosus GG, in particular Lactobacillus rhamnosus DSM 32550, is preferred for co-administration.

For co-administration, including simultaneous administration/delivery/consumption, the vitamin and the probiotic can be in the same compartment, or in separate compartments. For example, the vitamin and the bacteria can be in the same tablet/pill, or in the same sachet. Alternatively, the vitamin and the bacteria can be in separate tablets/pills, or in different sachets. In the latter case, an administration/delivery/consumption within 24 hours is still considered a co-administration.

Lactobacillus rhamnosus is known to improve the digestive health and to positively modulate the immune system. A number of studies have shown that the population of Lactobacillus rhamnosus in the gut microbiome is decreased when an animal, preferably a human is suffering from a particular disease/adverse condition as compared to the population present in the animal not suffering from that particular disease/adverse condition. However, none of these studies have suggested a method of how to increase the population of Lactobacillus rhamnosus, thus alleviating at least one of the symptoms of the disease/adverse condition. It has been found, in accordance with this invention, that direct delivery of vitamin B2 to the large intestine of an animal, preferably a human, can increase the population of Lactobacillus rhamnosus in the large intestine.

In a preferred embodiment, the animal (including a human) is experiencing a condition selected from the group consisting of: gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, and cancer.

Preferably, the Lactobacillus rhamnosus to be boosted is Lactobacillus rhamnosus GG (e.g. Lactobacillus rhamnosus DSM 32550). Thus, in one embodiment, the vitamin B2 is for use in increasing the abundance of a Lactobacillus rhamnosus GG (preferably Lactobacillus rhamnosus DSM 32550) in the large intestine of an animal.

In another aspect, the present invention relates to an oral formulation comprising vitamin B2 and Lactobacillus rhamnosus. Preferably, the oral formulation is a delayed-release formulation.

In yet another aspect, the present invention relates to a method of increasing the abundance of Lactobacillus rhamnosus in the intestine, preferably the large intestine, of an animal, comprising administering to the animal an effective dose of vitamin B2. The method is for improving intestinal health in an animal, wherein said improvement comprises increasing the abundance of Lactobacillus rhamnosus, preferably Lactobacillus rhamnosus GG (in particular, Lactobacillus rhamnosus DSM 32550), in the large intestine. Preferably, the animal is a human.

Preferably, the vitamin B2 is delivered directly to the large intestine. Delivery to the large intestine can be achieved by administering the vitamin B2 as a delayed-release formulation.

In one embodiment, Lactobacillus rhamnosus is co-administered with the vitamin B2. Preferably, an effective dose of a Lactobacillus rhamnosus GG (such as Lactobacillus rhamnosus DSM 32550) is co-administered.

The methods of the invention can be used to treat, prevent, and/or lessen the symptoms of gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, and cancer.

In a further aspect, the present invention relates to the use of vitamin B2 for increasing the abundance of Lactobacillus rhamnosus in the intestine of an animal, preferably a human, wherein said use comprises delivering the vitamin B2 to the large intestine. Preferably, the use comprises delivering/administering the vitamin B2 to the large intestine by a delayed-release formulation. In one embodiment, the use comprises administering to the animal a formulation comprising a vitamin B2 dose of up to 200 mg/day, preferably 5-100 mg/day, more preferably 10-50 mg/day. The vitamin B2 may be co-administered with Lactobacillus rhamnosus. Preferably, the animal, including a human, is experiencing a condition selected from the group consisting of: gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, and cancer. Preferably, the Lactobacillus rhamnosus is Lactobacillus rhamnosus GG. Lactobacillus rhamnosus DSM 32550 is particularly preferred.

A preferred way of (co-) administration is oral administration.

### Definitions

As used throughout, the following definitions apply:

The term “vitamin B2”, which is used interchangeably with “riboflavin”, includes riboflavin and esters thereof, in particular riboflavin-5′-phosphate, and other pharmaceutically acceptable forms.

To “increase the abundance” of Lactobacillus rhamnosus means to increase the level (or the amount, or number, or the population size) of Lactobacillus rhamnosus compared to the respective control (i.e., the level/amount/number/population size of Lactobacillus rhamnosus without the addition of vitamin B2).

The term “intestine” (or “gut”) as used herein refers to the portion of the gastrointestinal tract consisting of the small intestine and the large intestine. The “large intestine” (intestinum crassum) is the lower part of the gastrointestinal tract and is also referred to herein as “colon”.

“Direct delivery” or “directly delivered” means that the vitamin is formulated in a manner such that the vitamin is not absorbed in the stomach and/or small intestine; rather the vitamin is made available in the distal intestinal tract, preferably the large intestine (colon), where it is available to the microbiome. The vitamin is not part of a person's usual daily nutritional requirements (generally obtained through diet and conventional vitamin supplementation) and is administered in excess thereof. For human use, the preferred method according to the present invention is through a form which delays release until the large intestinal tract (colon) is reached. Alternatively, a large enough dose can be administered, so that only a portion of the administered vitamin is absorbed in the proximal small intestine, and the remainder, which is an effective dose, is available to the large intestinal tract; although not preferred, the latter method of delivery can be used for humans as well.

A used herein, “delayed release” refers to the release of the vitamin and/or the probiotic at a time later than immediately after administration. Preferably, “delayed release” means delivery of the vitamin (and/or probiotic), upon oral administration, to the large intestine (colon) in a delayed manner relative to an immediate release formulation.

An “enteric layer” or “enteric coating” is a layer surrounding a core, wherein the core comprises the active agent and the layer confers resistance to gastric juice.

“Prevent” can include lessening the risk of an adverse condition occurring, lessening the symptoms of an adverse condition, lessening the severity of an adverse condition, and prolonging the time for occurrence of an adverse condition.

“Oral formulation” means that the vitamin and/or probiotic is formulated for oral administration/consumption.

“Co-administering” or “co-administration” means that the vitamin and/or the probiotic is delivered/administered/consumed simultaneously (i.e., together), or separately but within a time frame of 24 hours. The vitamin can be delivered/administered/consumed first; likewise, the probiotic can be delivered/administered/consumed first.

Lactobacillus rhamnosus was recently renamed Lacticaseibacillus rhamnosus; both names are used interchangeably herein, and both can be abbreviated as L. rhamnosus.

### Doses

The vitamin B2 can be administered in an amount such that its local concentration in the colon is at least 0.001 g/L, preferably at least 0.01 g/L more preferably at 0.02 g/L. Preferred local concentrations in the colon range from about 0.001 g/L to about 0.5 g/L or from about 0.005 g/L to about 0.2 g/L, preferably about 0.01 to about 0.02 g/L. Specific dosages per day can range up to 200 mg/day, preferably 5-100 mg/day, more preferably from 10-50 mg/day.

The dosage of the probiotic can be up to 5E±10 cfu/day. Preferably, the dosage range of the probiotic is from 1E±08 to 1E±10 cfu/day, more preferably from 1E±09 to 5E±10 cfu/day.

### Formulations

The vitamin (and, where the vitamin is co-administered with the probiotic, preferably also the probiotic) is preferably present in a formulation which allows the vitamin (and/or probiotic) to be available predominantly in the large intestine.

Oral formulations are preferred. Other formulations include non-oral routes, such as via suppositories or injections.

For human use, the preferred method is through a delayed-release form which delays delivery until the intestinal tract is reached. For non-human animals, a preferred delivery includes a method of administering a large enough dose so that only a portion of the vitamin delivered is absorbed in the stomach, and the remainder, which is an effective dose, is available to the intestinal tract; although not preferred, this method of delivery can be used for humans as well.

Delayed-release formulations are known in the art. Preferably, the delayed-release formulations have an enteric coating (also referred to as enteric layer).

In one embodiment of the present invention, the vitamin (and, where the vitamin is co-administered with the probiotic, preferably also the probiotic) is in a formulation comprising an enteric capsule, filled with a composition comprising the vitamin. The enteric capsule confers resistance against the acidic environment of the stomach. For example, soft gel formulations may deliver the active agent in solution and yet offer advantages of solid dosage forms.

In another embodiment, the formulation is a tablet comprising (i) a core comprising the vitamin (and/or the probiotic), and (ii) a delayed-release coating such as an enteric coating. This may be a hard gel capsule.

Alternatively, a matrix-based delivery system can be used for direct colon delivery. Matrix based systems have no discrete layer of coating material, but the active agent is more or less homogenously distributed within the matrix. Further, there are colon-release systems that embed the active agent in e.g. in a fiber matrix (enzyme-triggered) and an enteric coating on top.

The release of the drug may be delayed until the small intestine. In another embodiment, the release of the drug is delayed until the distal small intestine. In yet another, preferred embodiment, the release of the drug is delayed until the colon (large intestine).

In a preferred embodiment for humans, the vitamin is formulated in a solid dosage form for oral administration. The formulation may be in the form of a capsule, pellet, bead, sphere, mini spheres, tablet, mini tablet, or granule, optionally coated with a delayed release coating that prevents the release of the active agent before the small intestine, preferably before the colon.

Coating, or matrix materials for the delayed release of the vitamin and/or probiotic, in particular for targeted release in the ileum or the large intestine upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating materials from different categories are commonly used in combinations. Coating materials of the different categories for targeting to the large intestine have been reviewed for example in Bansal et al. (Polim. Med. 2014, 44, 2, 109-118). In one embodiment of the present invention, the delayed-release coating comprises at least one component selected from coating materials that disintegrate pH-dependently, coating materials that disintegrate time-dependently, coating materials that disintegrate due to enzymatic triggers in the intestinal environment (e.g., in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit® L-100, Eudragit® L12.5), poly(methacrylic acid, methyl methacrylate) 1:2 (Eudragit® S-100, Eudragit® S12,5, and Eudragit® FS30D). Coating materials that disintegrate time-dependently include Eudragit® RL, Eudragit®RS, and ethylcellulose. Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

The following non-limiting examples are presented to better illustrate the invention.

### EXAMPLES

The aim of this study was to investigate the effect of vitamin B2 on bacteria of the species Lactobacillus rhamnosus in the human gut microbiota.

### Materials and Methods

### Design of the Batch-Fermentation Experiment (Colon Model)

A short-term batch-fermentation experiment was carried out by ProDigest, consisting of a colonic incubation of a representative dose of a selected vitamin with a representative bacterial inoculum under simulated conditions for the proximal large intestine. In the current experiment, the bacterial inocula were derived from fresh fecal samples of six different healthy adult donors. Incubations were performed as described previously (Van den Abbeele, P.; Taminiau, B.; Pinheiro, I.; Duysburgh, C.; Jacobs, H.; Pijls, L.; Marzorati, M. Arabinoxylo-Oligosaccharides and Inulin Impact Inter-Individual Variation on Microbial Metabolism and Composition, Which Immunomodulates Human Cells. J. Agric. Food Chem. 2018, 66, 1121-1130). At the start of the short-term colonic incubation, fresh fecal material from six healthy human donors was collected and upon preparation of an anaerobic fecal slurry, this slurry was inoculated at 10 vol % in a SHIME nutritional medium containing basal nutrients containing 3.5 g/L K2HPO4, 10.9 g/L KH2PO4, 2 g/L NaHCO3 (Chem-lab NV, Zedelgem, Belgium), 2 g/L yeast extract, 2 g/L peptone (Oxoid, Aalst, Belgium), 1 g/L mucin (Carl Roth, Karlsruhe, Germany), 0.5 g/L L-cysteine and 2 mL/L Tween80 (Sigma-Aldrich, Bornem, Belgium). All test ingredients (i.e., probiotic strain, vitamin B2) were also added to the SHIME medium. Furthermore, M-SHIME® technology was incorporated in the current experiment by adding mucin-covered microcosms (modeling the mucus of the colon) to the incubations as described previously (Van den Abbeele, P., et al. (2013). Butyrate-producing Clostridium cluster XIVa species specifically colonize mucins in an in vitro gut model. The ISME Journal 7 (5), 949-961). Incubations were performed for 48 h, at 37° C., under shaking (90 rpm) and anaerobic conditions.

In this study, the Lactobacillus rhamnosus GG equivalent Lactobacillus rhamnosus DSM 32550 (Biocare Copenhagen) was added to the colon model containing the donor samples, alone or in combination with vitamin B2 (see Table 1). After 48 h of incubation, the abundance of the added strain was analyzed using primers that are specific for Lactobacillus rhamnosus GG and its equivalent Lactobacillus rhamnosus DSM 32550. Experiments were performed in single repetition.

Lactobacillus rhamnosus DSM 32550 has a genomic sequence which is 99.99% identical to the genomic sequence of LGGR. It can therefore be considered that L. rhamnosus DSM 32550 is identical or equivalent to LGG® for practical purposes. In the Examples herein, Lactobacillus rhamnosus DSM 32550 will thus be referred to as a Lactobacillus rhamnosus GG strain.

TABLE 1
Experiment layout

| | Do- | Do- | Do- | Do- | Do- | Do- |
|---|---|---|---|---|---|---|
| Test ingredient | nor 1 | nor 2 | nor 3 | nor 4 | nor 5 | nor 6 |
| Lactobacillus | x | x | x | x | x | x |
| rhamnosus GG | | | | | | |
| only (Control) | | | | | | |
| Lactobacillus | x | x | x | x | x | x |
| rhamnosus | | | | | | |
| GG + Vitamin B2 | | | | | | |

The Lactobacillus rhamnosus GG equivalent strain Lactobacillus rhamnosus DSM 32550 was added to the SHIME medium as an overnight grown culture at a concentration of 1*109 CFU. The probiotic strain was either supplemented alone or in combination with vitamin B2. Vitamin B2 (Riboflavin TG, DSM) was added at a concentration of 0.01667 g/L which translates into a 10 mg dose, given that the colon volume is about 600 ml.

Analysis of the Lactobacillus rhamnosus GG Level

qPCR was performed on putative phage tail protein gene (NCBI reference NC_013198) to monitor the level of the supplemented probiotic Lactobacillus rhamnosus GG strain within the gut microbiome, as described previously (Brandt, Alatossava (2003)-Specific identification of certain probiotic Lactobacillus rhamnosus strains with PCR primers based on phage-related sequences). Samples were analyzed from the mucus environment following 48 h of incubation.

### Statistics

Statistical analysis was performed to investigate the average effect of the test products. For this purpose, the average over the six donors was calculated for each endpoint. Paired t-tests were conducted to evaluate the potential effect of the test products as compared to the control as well as to compare the different test products with each other. For the microbial community composition, statistical tests were performed on the log-transformed data (to make them normally distributed). Differences were considered statistically significant if the p-value was less than 0.05.

### Results

Supplementation of Vitamin B2 Increased the Level of Lactobacillus rhamnosus GG

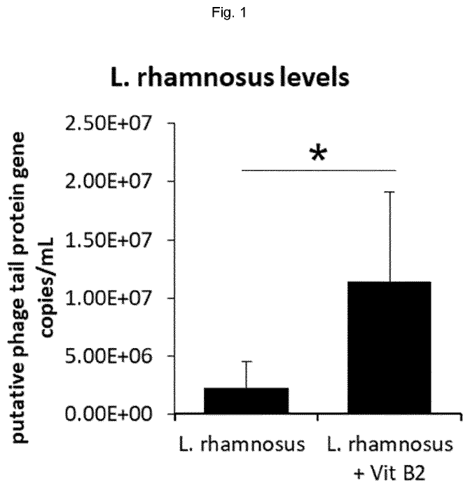

As shown in FIG. 1, around three times as many copies of the Lactobacillus rhamnosus GG specific putative phage tail protein gene were detected in the mucus environment by qPCR analysis when vitamin B2 was co-supplemented with Lactobacillus rhamnosus GG. Hence, the addition of vitamin B2 significantly increased the levels of Lactobacillus rhamnosus GG in the colon model, as compared to the respective control.

1. Vitamin B2 for use in increasing the abundance of Lactobacillus rhamnosus in the intestine of an animal, preferably a human, wherein said use comprises delivering the vitamin B2 to the large intestine.

2. Vitamin B2 for the use according to claim 1, wherein the vitamin B2 is delivered to the large intestine by a delayed-release formulation.

3. Vitamin B2 for the use according to claim 1, wherein said use comprises administering to the animal a formulation comprising a vitamin B2 dose of up to 200 mg/day, preferably 5-100 mg/day, more preferably 10-50 mg/day.

4. Vitamin B2 for the use according to claim 1, wherein the vitamin B2 is co-administered with Lactobacillus rhamnosus.

5. Vitamin B2 for the use according to claim 1, wherein the animal, including a human, is experiencing at least one condition selected from the group consisting of: gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, and cancer.

6. Vitamin B2 for the use according to claim 1, wherein the Lactobacillus rhamnosus is Lactobacillus rhamnosus GG.

7. An oral formulation comprising vitamin B2 and Lactobacillus rhamnosus.

8. The oral formulation according to claim 7 which is a delayed-release formulation.

9. A method of increasing the abundance of Lactobacillus rhamnosus in the intestine of an animal, preferably a human, comprising administering to the animal a formulation comprising an effective dose of vitamin B2.

10. The method according to claim 9, wherein the animal is a human and the vitamin B2 is delivered to the large intestine.

11. The method according to claim 9, wherein the vitamin B2 is delivered by a delayed-release formulation.

12. The method according to claim 9, wherein the vitamin B2 is co-administered with Lactobacillus rhamnosus.

13. The method according to claim 9, which is a method of treating, preventing, or lessening the symptoms of gastrointestinal infections and diarrhea, antibiotic and Clostridium difficile associated diarrhea, irritable bowel syndrome, inflammatory bowel disease, respiratory tract infections, allergy, cardiovascular diseases, nonalcoholic fatty liver disease, nonalcoholic steatohepatitis, cystic fibrosis, or cancer in an animal, including a human, in need thereof.

14. The method according to claim 9, wherein the Lactobacillus rhamnosus is Lactobacillus rhamnosus GG.
